(19) United States
(12) Patent Application Publication
(10) Pub. No.: US 2018/0209981 A1
(43) Pub. Date: Jul. 26, 2018

(54) WBP2 AS A CO-PROGNOSTIC FACTOR WITH HER2 FOR STRATIFICATION OF PATIENTS FOR TREATMENT

(71) Applicant: National University of Singapore, Singapore (SG)

(72) Inventor: Yoon Pin LIM, Singapore (SG)

(21) Appl. No.: 15/746,506

(22) PCT Filed: Jul. 19, 2016
(86) PCT No.: PCT/SG2016/050341

§ 371 Date: Jan. 22, 2018

(30) Foreign Application Priority Data
Jul. 23, 2015 (SG) 10201505756X

(51) Int. Cl. G01N 33/574

(52) U.S. Cl. CPC G01N 33/57415; G01N 2333/4703; G01N 2333/71

(57) ABSTRACT

The present invention provides a method for the prognosis of overall survival, cancer recurrence or response to treatment for a patient suffering from cancer, the method comprising: (a) examining a sample from the patient to determine whether the patient is human epidermal growth factor receptor 2 (HER2) positive or negative; and (b) measuring WW domain-binding protein 2 (WBP2) levels in the patient's sample, wherein a result in step (a) and a result in step (b) provides a prognosis of overall survival, cancer recurrence or response to treatment for the patient. The present invention also provides a kit carrying out the method of the present invention.

### FIELD

The present invention relates to a prognostic method, system and kit for cancer, in particular breast cancer. The present invention also relates to the stratification of a population of patients for cancer treatment, in particular breast cancer treatment. Further the present application relates to the identification of cancer markers for use in a method, system and kit for the prognosis of cancer, in particular breast cancer.

### BACKGROUND

The following discussion of the background to the invention is intended to facilitate an understanding of the present invention. However, it should be appreciated that the discussion is not an acknowledgment or admission that any of the material referred to was published, known or part of the common general knowledge in any jurisdiction as at the priority date of the application.

Worldwide breast cancer is the second most common type of cancer and one of the most common causes of cancer death in humans. It is the most common cancer in women and makes up a third of cancer occurrence of women in the US. Common tests that provide information to assist in the diagnosis or prognosis of breast cancer include mammograms and tissue biopsy followed by combinations of histological examination, immune-histochemical detection with antibodies to estrogen receptor (ER), progesterone receptor (PR) and/or HER2/neu proteins.

Current treatment of breast cancer includes surgery, chemotherapy, radiation therapy and immunotherapy. Targeted therapy such as HER2/neu antibody (i.e. Herceptin (Trastuzumab)) first became available in the late 1990's. Later developed HER2/neu antibodies include Pertuzumab and Lapatinib.

HER2 is a cancer biomarker for aggressive cancer where overexpression of HER2 occurs in approximately 30% of breast cancer. Over expression of HER2 also occurs in ovarian, stomach, gastric and uterine cancers. The HER2 receptor protein is a target for HER2 antagonists such as Trastuzumab, Pertuzumab and Lapatinib. Thus the first priority for eligibility for therapeutic use of HER2 antagonists is the demonstration, for example by immunocytochemistry, of the over expression of the membrane domain of HER2. However, not all patients with HER2 positive cancers respond to treatment and some HER2 positive cancers are self-limiting even without treatment. This suggests that there are subpopulations of HER2 positive cancers that are more aggressive and/or intrinsically resistant to treatment, particularly Herceptin treatment.

Testing for HER2 includes but is not limited to fluorescence in situ hybridization (FISH) to detect the number of HER2 gene present in a sample and ImmunoHistoChemistry (IHC) to detect the amount of HER2 protein in a sample. The latter method is however semi-quantitative.

Therefore there is a need to determine cancer markers and to find improved methods, systems and kits which allow continuous more accurate quantification with increased sensitivity for the prognosis of cancer, in particular breast cancer or other cancer types such as gastric cancer that demonstrate amplification or over expression of the ERBB2 gene that expresses HER2.

There is a need for alternative methods and kits for stratifying cancer patients to ameliorate at least one of the problems mentioned above

### SUMMARY

It is an object of the present invention to provide improved methods and kits in accordance with the present invention.

Accordingly, an aspect of the present invention, provides a method for the prognosis of overall survival, cancer recurrence or response to treatment for a patient suffering from cancer, the method comprising: (a) examining a sample from the patient to determine whether the patient is human epidermal growth factor receptor 2 (HER2) positive or negative based on a predetermined level of HER2; and (b) measuring WW domain-binding protein 2 (WBP2) levels in the patient's sample, wherein a result in step (a) and a result in step (b) provides a prognosis of overall survival, cancer recurrence or response to treatment for the patient.

Another aspect of the invention provides a kit for identifying in a sample the amount of human epidermal growth factor receptor 2 (HER2) and the amount of WW domain-binding protein 2 (WBP2), the kit comprising: (a) at least one first probe adapted to detect and measure a human epidermal growth factor receptor 2 (HER2) level in the sample to determine whether the sample is HER2 positive or HER2 negative; and (b) at least one second probe adapted to detect and measure WW domain-binding protein 2 (WBP2) levels in the sample.

Another aspect of the invention provides an in vitro method for determining the prognosis of overall survival, cancer recurrence or response to treatment, the method comprising: (a) measuring the level of human epidermal growth factor receptor 2 (HER2) in a sample; (b) measuring the level of WW domain-binding protein 2 (WBP2) in the sample, and (c) determining whether the level of HER2 and WBP2 are above or below a predetermined level wherein a result in step (c) provides a prognosis of overall cancer survival, cancer recurrence or response to cancer treatment.

Other aspects of the invention will become apparent to those of ordinary skill in the art upon review of the following description of specific embodiments of the invention in conjunction with the accompanying figures.

### BRIEF DESCRIPTION OF THE DRAWINGS

The invention will now be described, by way of example only, with reference to the accompanying drawings, in which:

FIG. 1 provides Kaplan-Meier survival curves for an analysis involving more than 200 clinical specimens. (A) Overall survival in number of years depend on WBP2 and HER2 status (n=221); (B) Disease free survival in number of years depend on WBP2 and HER2 status. (n=221); (C) Kaplan-Meier survival analysis for overall survival depend on HER2 status; (D) and disease free survival depend on HER2 status; (E) Correlation of the amount of HER2 expressed with the amount of WBP2 in the nucleus; (F) Correlation of the amount of HER2 expressed with the amount of WBP2 in the cytoplasm.

FIG. 2 provides an Immunoblot analysis (A) showing that WBP2 mediates EGF/HER2 signalling and supports WBP2 as a potential predictor of response to drugs that target EGFR/HER2. Her2 signals through WBP2, because of this, blocking Her2 when WBP2 activity is aberrant will not be effective in killing cancer cells because the aberrant activity of WBP2 will drive cancer growth. This means that aberrant levels of WBP2 may predict response to Herceptin. HER2 was knocked down in human breast cancer SK-BR-3 cells by transfection of HER2 siRNA. Luciferase siRNA was used as negative control. Cells were treated with 50 ng/ml EGF for 10 min after 24 hr serum starvation. Cell lysates were immunoprecipitated (IP) with anti-WBP2 antibody and phosphorylation of endogenous WBP2 were analysed by Western blot (IB) using anti-phosphotyrosine (PY20). Phosphorylation of HER2 and EGFR were analysed by Western blot (IB) with indicated antibodies. β-tubulin was used as a protein loading control. HER2 was knocked down in human breast cancer cells, SK-BR-3(B) and ZR-751(C) by transfection of HER2 siRNA. Luciferase siRNA was used as negative control. Cells were treated with 50 ng/ml EGF for 10 min after 24 hr serum starvation. Cell lysates were immunoprecipitated(IP) with anti-WBP2 antibody and phosphorylation of endogenous WBP2 were analyzed by Western blot (IB) using anti-phosphotyrosine (PY20) and anti-WBP2 antibodies. Phosphorylation of HER2 was analyzed by Western blot (IB) with indicated antibodies. β-tubulin was used as protein loading control.

FIG. 3 Trastuzumab dose-response with WBP2 expression level on cell proliferation. WBP2 was overexpressed in BT-474 using WBP2 expressing lentivirus (A and B) and WBP2 was knocked-down using two different shRNA targeting WPB2 in SK-BR-3 (C and D) and two different siRNA targeting WBP2 in ZR-75-30 (E and F). Cells were plated on 96-well plates for 2D culture (A and C) or 96-well ultra-low attachment plates for 3D culture (B and D) at 10,000 cells per well. After 3 days (SK-BR-3) or 5 days (BT-474) incubation with trastuzumab, the viability of cells were measured by using Cell Titer 96 aqueous non-radioactive cell proliferation assay. Viability of cells was calculated as fold change compared to trastuzumab-untreated control cells. The data represent mean±SD. Statistical significance was determined by Student's t-test (* or +P<0.05; ** or ++P<0.01; *** or +++P<0.001 vs. vector or control).

FIG. 4 Trastuzumab dose-response with WBP2 expression on HER2 level and downstream signalling pathway. WBP2 was overexpressed in BT-474 using WBP2 expressing lentivirus (A) and WBP2 was knocked down using two different shRNA targeting WPB2 in SK-BR-3 (B) and two different siRNA targeting WBP2 in ZR-75-30 (C). Cells were treated with different concentration of trastuzumab (0, 1, 10, 100 μg/ml) for 3 days (SK-BR-3) or 5 days (BT-474 and ZR-75-30). Expression of Her2, WBP2 and β-tubulin were analyzed by Western blot.

FIG. 5 Effect of WBP2 expression on Trastuzumab-treatment in vivo. All animal housing and handling procedures were in accordance with institutional guidelines at National University of Singapore. For the Xenograft model, 5-week-old female Athymic Nude mice (n=6-7, In Vivos, Singapore) were implanted with 0.72 mg 60 day release 17β-estradiol pellets (Innovative Research, Sarasota, Fla., USA) and, after 2 days, BT-474 control (vector) or WBP2 overexpressing cells (1×107 in 200 μl of DPBS and Matrigel 1:1 mixture) were injected subcutaneously into a mouse mammary fat pad. When the tumors reached 150-200 mm3, the mice were divided into groups, keeping average tumor size similar between groups, and treated with trastuzumab (10 mg/kg, Roche) or PBS (control) by intraperitoneally (IP) twice weekly for three weeks. The tumor size was measured twice weekly with calipers and tumor volumes calculated as follow: volume=(width2×length)/2. The data represent mean±SEM. Statistical significance was determined by Mann-Whitney test.

The accompanying drawings are not to be understood as superseding the generality of the preceding description of the invention.

### DETAILED DESCRIPTION

Particular embodiments of the present invention will now be described with reference to the accompanying drawings. The terminology used herein is for the purpose of describing particular embodiments only and is not intended to limit the scope of the present invention. Additionally, unless defined otherwise, all technical and scientific terms used herein have the same meanings as commonly understood by one or ordinary skill in the art to which this invention belongs.

The present technology relates to the correlation of human epidermal growth factor receptor 2 (HER2) positive or negative determination and WW domain-binding protein 2 (WBP2) levels with the overall survival, cancer recurrence or response to cancer treatment for a patient suffering from cancer, in particular breast cancer.

Accordingly, an aspect of the present invention, provides a method for the prognosis of overall survival, cancer recurrence or response to cancer treatment for a patient suffering from cancer, the method comprising: (a) examining a sample from the patient to determine whether the patient is human epidermal growth factor receptor 2 (HER2) positive or negative based on a predetermined level of HER2; and (b) measuring WW domain-binding protein 2 (WBP2) levels in the patient's sample, wherein a result in step (a) and a result in step (b) provides a prognosis of overall survival, cancer recurrence or response to treatment for the patient.

As used herein the term ‘prognosis of overall survival’ refers to determining roughly how long a patient is likely to live based on the amount of HER2 and WBP2 in the sample from the patient. In various embodiments the status of whether the patient is living or dead may be measured over the course of from 1 year, or from 2 year, or from 3 years, or from 4 years, or from 5 years, or from 6 years, or from 7 years, or from 8 years, or from 9 years, or from 10 years, or from 11 years, or from 12 years or more.

As used herein the term ‘prognosis of cancer recurrence’ refers to determining if a patient is likely to contract cancer again at a later time after the cancer is observed or considered to have gone from the patient based on the amount of HER2 and WBP2 in the sample from the patient. In various embodiments the development of cancer and whether a patient contracts cancer again may be stratified into subgroups for example: no cancer, local cancer that may be sub classified based on the size of the tumour, metastasis, or death and in various categories the rate of the development of one or more of these subgroups over time. In various embodiments the status of whether the patient has cancer recurrence may be measured over the course of from 1 year, or from 2 year, or from 3 years, or from 4 years, or from 5 years, or from 6 years, or from 7 years, or from 8 years, or from 9 years, or from 10 years, or from 11 years, or from 12 years or more.

As used herein the term ‘prognosis of response to cancer treatment’ refers to determining if a patient is likely to respond positively to a cancer treatment based on the amount of HER2 and WBP2 in the sample from the patient. Wherein a patient responds positively to a cancer treatment where the cancer is cured, prevented or slowed down (lessened) over time. In various embodiments the status of whether the patient responds positively to a cancer treatment may be measured over the course of from 3 weeks, or from 25 weeks, or from 1 year, or from 2 years, or from 3 years, or from 4 years, or from 5 years, or from 6 years, or from 7 years, or from 8 years, or from 9 years, or from 10 years, or from 11 years, or from 12 years or more.

As used herein the term “sample” refers to any tissue or fluid obtained from an individual, for example via a biopsy. A “sample” includes, but is not limited to, e.g., plasma, serum, spinal fluid, lymph fluid, the external sections of the skin, respiratory, intestinal, and genitourinary tracts, tears, saliva, blood cells, organs, tissue including breast tissue and samples of in vitro cell culture constituents. The sample may be present on a tissue array or may comprise a whole tissue section.

As used herein the term ‘patient’ refers to an animal such as a mammal that is suspected of having or suffering from cancer. In various embodiments this may include animals at risk of having cancer, animals that have cancer or animals that have had cancer in the past. In various embodiments the patient comprises a human.

In various embodiments a patient is identified by conducting a mammogram. Whereby any observed mass is sufficient for the patient to be suspected of having or suffering from cancer. In various embodiments a patient is identified by conducting a tissue biopsy wherein the sample is classified as atypical, neoplasia, carcinoma or dysplasia is sufficient for the patient to be suspected of having or suffering from cancer. Any organ in the body can be biopsied using a variety of techniques, some of which require major surgery (e.g., staging splenectomy for Hodgkin's disease), while others do not even require local anesthesia (e.g., fine needle aspiration biopsy of thyroid, breast, lung, liver, stomach etc).

HER2 is a cancer biomarker where overexpression of HER2 occurs in approximately 30% of breast cancer. Any method known in the art for determining whether the patient is human epidermal growth factor receptor 2 (HER2) positive or HER2 negative would be suitable for use in the method described herein. HER2 is a target for treatments which include but are not limited to Trastuzumab, Pertuzumab and Lapatinib. Methods for testing patients as to whether they are HER2 positive or negative include but are not limited to fluorescence in situ hybridization (FISH) to detect the number of HER2 gene present in a patient's sample and ImmunoHistoChemistry (IHC) to detect the amount of HER2 protein in a patient's sample. IHC uses an antibody to evaluate HER2 protein expression. Methods and their associated techniques, such as FISH and IHC, for determining whether a patient is HER2 positive or negative are known in the art. IHC has a scoring system which is used to determine whether a patient is HER2 positive or HER2 negative. This is based on a predetermined set level of HER2 gene expression. A patient's sample having an IHC score of the predetermined level of about 1-2 or more will indicate that the patient is HER2 positive while an IHC score of less than the predetermined level of about 1-2 will indicate that the patient is HER2 normal or HER2 negative. Such patients still have HER2 expression but they are considered to be in the normal range. For IHC techniques see, e.g. Dabbs D. J., 2006 (2nd Edition): “Diagnostic Immunohistochemistry”. It is appreciated that depending on the method adopted, the scoring system may differ and the predetermined set level may adjust to the scoring system.

Another way of examining a sample from the patient to determine whether the patient is human epidermal growth factor receptor 2 (HER2) positive or negative based on a predetermined level of HER2 is in situ hybridization (ISH). ISH determines the number of HER2 copies using a DNA probe coupled to a fluorescent, chromogenic, or silver detection system (ie, FISH, CISH, or SISH), or a combination of CISH and SISH systems (bright-field double ISH (BDISH) or dual-hapten, dual-colour ISH (DDISH)). ISH is conducted using a single probe to enumerate HER2 copies per nucleus only or as a dual-probe technique where hybridization of a chromosome 17 centromere probe (chromosome enumeration probe 17, CEP17) allows determination of the HER2:CEP17 ratio. The two-probe approach may be performed as a dual-colour technique, with co-hybridisation of the two probes on the same slide, or as a monochrome assay where each probe is used on sequential slides. The HER2:CEP17 ratio is sometimes regarded as a better reflection of HER2 amplification status than mean HER2 copy number, as the latter is also dependent on the mitotic index of the tumour, section thickness, nuclear truncation effects, and abnormal chromosome copy number.

The most accepted predetermined levels of determining HER2 negative or HER2 positive are issued by the USA food and drug administration (FDA) or the American Society of Clinical Oncology/College of American Pathologists (ASCO/CAP). Both are summarised in table 1.

TABLE 1
USA food and drug administration or the American Society of Clinical Oncology/College of
American Pathologists predetermined level for HER2 status determination by IHC or FISH.

| | | | FISH | | | |
|---|---|---|---|---|---|---|
| | | | HER2 copies | | | |
| | | | average/nucleus | | HER:CEP17 ratio | |
| | IHC | | | ASCO/ | | ASCO/ |
| | FDA | ASCO/CAP | FDA | CAP | FDA | CAP |
| Negative | 0-1+ | 0-1+ | ≤4.0 | <4.0 | ≤2.0 | <1.8 |
| Equivocal | — | 2+ (non-uniform or weak | — | 4.0-6.0 | — | 1.8-2.2 |
| | | complete membrane staining | | | | |
| | | in ≥10% tumor cells, or | | | | |
| | | intense, complete membrane | | | | |
| | | staining in ≤30% invasive | | | | |
| | | tumour cells) | | | | |
| positive | 2+ (weak-to-moderate | 3+ (uniform intense | >4.0 | >6.0 | ≥2.0 | >2.2 |
| | complete membrane staining | membrane staining of >30% | | | | |
| | in >10% of tumour cells), | invasive tumour cells) | | | | |
| | 3+ (strong complete membrane | | | | | |
| | staining in >10% of tumour | | | | | |
| | cells). Patients with IHC | | | | | |
| | 2+/ISH− tumours are not | | | | | |
| | eligible for Trastuzumab | | | | | |
| | treatment to date | | | | | |

Not all patients with HER2 positive breast cancer respond to treatment and some HER2 positive breast cancers are self-limiting even without treatment. This suggests that there are subpopulations of HER2 positive breast cancers that are more aggressive and/or intrinsically resistant to treatment.

WBP2 is a mediator of EGFR (epidermal growth factor receptor), ER (estrogen receptor) and Wnt signalling (Lim S K et al. (2011)) in breast cancer cells. WBP2 and proteins that regulate its expression can be used to predict response to drugs. WBP2 levels in a sample may be measured by detecting the amount of nuclear and/or cytoplasmic/non-nuclear WBP2 proteins using antibodies or aptamers, or detecting genomic amplification using DNA probes. FISH and IHC may be used to determine whether a patient has high or low WBP2 levels. The Sequence of WBP2 protein is set forth in amino acid sequence SEQ ID NO. 1:

MALNKNHSEG GGVIVNNTES ILMSYDHVEL TFNDMKNVPE
AFKGTKKGTV YLTPYRVIFL SKGKDAMQSF MMPFYLMKDC
EIKQPVFGAN YIKGTVKAEA GGGWEGSASY KLTFTAGGAI
EFGQRMLQVA SQASRGEVPS GAYGYSYMPS GAYVYPPPVA
NGMYPCPPGY PYPPPPPEFY PGPPMMDGAM GYVQPPPPPY
PGPMEPPVSG PDVPSTPAAE AKAAEAAASA YYNPGNPHNV
YMPTSQPPPP PYYPPEDKKT Q

WBP2 is a biomarker that can be used to stratify HER2 positive breast cancers into lowly and highly aggressive cases for treatment and surveillance.

As used herein the term ‘lowly aggressive cancers’ refer to patients with cancer that are less likely to die or have the recurrence of cancer over a period of time. In various embodiments the cancer status of the patient may be measured over the course of from 1 year, or from 2 years, or from 3 years, or from 4 years, or from 5 years, or from 6 years, or from 7 years, or from 8 years, or from 9 years, or from 10 years, or from 11 years, or from 12 years or more.

As used herein the term ‘highly aggressive cancers’ refer to patients with cancer that are more likely to die or have the recurrence of cancer over a period of time. In various embodiments the cancer status of the patient may be measured over the course of from 1 year, or from 2 years, or from 3 years, or from 4 years, or from 5 years, or from 6 years, or from 7 years, or from 8 years, or from 9 years, or from 10 years, or from 11 years, or from 12 years or more.

In various embodiments the results in steps (a) and (b) are compared to a set of predetermined expression level results from a comparison population.

The term “comparison population” as used herein refers to measurements of HER2 and WBP2 to determine the presence or amount in a sample taken from a plurality of individuals of a population. In various embodiments the plurality of individuals include at least five individuals however any number of individuals may be suitable including less or more than 5 individuals provided the individuals are at risk of having cancer, have cancer or have had cancer in the past. The measurements form a reference. In various embodiments the development of cancer over time in each of the individuals is measured over the course of from 1 year, or from 2 years, or from 3 years, or from 4 years, or from 5 years, or from 6 years, or from 7 years, or from 8 years, or from 9 years, or from 10 years, or from 11 years, or from 12 years or more. In various embodiments the development of cancer may be stratified into subgroups for example: no cancer, local cancer that may be sub classified based on the size of the tumour, metastasis, or death and in various categories the rate of the development of one or more of these subgroups. There are a range of methods that may be used to derive values for comparison populations that could be determined by a person skilled in the art based on the measurements of HER2 and WBP2 and the development of cancer.

In various embodiments the comparison population is stratified into a plurality of subgroups determining the aggressiveness of a cancer.

In various embodiments each subgroup is referenced from a reference group of HER2 negative patients comprising WBP2 expression below a predetermined level.

In various embodiment WBP2 expression below a predetermined level comprises an IHC score of 1 and below, while high WBP2 is IHC score of greater than 1.

As used herein the term “Predetermined level” refers to an assay cut off value that is used to assess prognostic, or therapeutic efficacy results by comparing the assay results against the predetermined level/cut off, where the predetermined level/cut off already has been linked or associated with various clinical parameters (for example, sub-division of disease/condition, severity of disease/condition, progression, non-progression, or improvement of disease/condition with treatment. The disclosure provides exemplary predetermined level/cut off. However, it would be appreciated that cut off values may vary depending on the nature of the assay (for example, antibodies employed, reaction conditions, sample purity, etc.). Furthermore, it would be appreciated that the disclosure herein may be adapted for other assays, such as immunoassays to obtain immunoassay-specific cut off values for those other assays based on the description provided by this disclosure. Whereas the precise value of the predetermined limit/cut off may vary between assays, the correlations as described herein should be generally applicable.

In various embodiments a sample with WBP2 expression levels above the predetermined level and a HER2 positive patient provides the prognosis that the patient has an approximate 4 to 5 times lower chance of overall survival compared to the reference group.

In various embodiments a sample with WBP2 expression levels below the predetermined level and a HER2 positive patient provides the prognosis that the patient has an approximate 1 to 2 times lower chance of overall survival compared to the reference group.

In various embodiments a sample with WBP2 expression levels above the predetermined level and a HER2 negative patient provides the prognosis that the patient has an approximate 2 to 3 times lower chance of overall survival compared to the reference group.

It is preferred that each subgroup is referenced from a reference group of HER2 negative patients, and wherein samples obtained from the reference group of HER2 negative patients have low WBP2 levels. Preferably, a sample with high WBP2 levels of a HER2 positive patient provides the prognosis that the patient has an approximate 4.5 times lower chance of overall survival compared to the reference group; a sample with low WBP2 levels of a HER2 positive patient provides the prognosis that the patient has an approximate 1.7 times lower chance of overall survival compared to the reference group.

In various embodiments a sample with WBP2 expression levels above the predetermined level and a HER2 positive patient provides the prognosis that the patient has an approximate 2 to 3 times higher chance of cancer recurrence compared to the reference group.

In various embodiments a sample with WBP2 expression levels below the predetermined level and a HER2 positive patient provides the prognosis that the patient has an approximate 1 times higher chance of cancer recurrence compared to the reference group.

In various embodiments a sample with WBP2 expression levels above the predetermined level and a HER2 negative patient provides the prognosis that the patient has an approximate 1 to 2 times higher chance of cancer recurrence compared to the reference group.

a sample with high WBP2 levels of a HER2 positive patient provides the prognosis that the patient has an approximate 2.6 times higher chance of cancer recurrence compared to the reference group; and a sample with low WBP2 levels of a HER2 positive patient provides the prognosis that the patient has an approximate 1.1 times higher chance of cancer recurrence compared to the reference group.

In various embodiments a sample with WBP2 expression levels above the predetermined level and a HER2 positive result predicts that the patient is likely to respond to treatment.

In various embodiments a sample with WBP2 expression levels below the predetermined level and a HER2 positive result predicts that the patient is less likely to respond to treatment.

WBP2 is a biomarker that can further stratify HER2 positive breast cancers into subgroups of poor responders and good responders to HER2 antagonist treatment. Whereby WBP2 expression levels above the predetermined level and a HER2 positive result indicate a patient would respond well to HER2 antagonist treatment. Conversely, WBP2 expression levels below the predetermined level and a HER2 positive result a patient would respond poorly or badly to HER2 antagonist treatment.

As used herein the term “Treatment” and “treat” and synonyms thereof refer to both therapeutic treatment and prophylactic or preventative measures, wherein the object is to cure, prevent or slow down (lessen) a cancer condition. In various embodiments the treatment reduces the amount of HER2 expressed in the cells of patients. Preferably, the treatment reduces the amount of HER2 expressed in the cells in a sample taken from a patients from HER2 positive to HER2 negative predetermined level.

In various embodiments the treatment comprises a HER2 antagonist.

In various embodiments the HER2 antagonist comprises Herceptin (Trastuzumab), Pertuzumab, Lapatinib, Lapatinib in combination with capecitabine, Trastuzumab emtansin, Ado-trastuzumab, Neratinib, Amrubicin, varlitinib or Dasatinib.

In various embodiments HER2 positive gastric cancer treatments include but are not limited to varlitinib, Herceptin (Trastuzumab), Pertuzumab and Lapatinib treatments. HER2 positive breast cancer treatments include but are not limited to varlitinib, Herceptin (Trastuzumab), Pertuzumab and Lapatinib treatments. In various embodiments HER2 positive cholangiocarcinoma treatments include but are not limited to varlitinib, Herceptin (Trastuzumab), Pertuzumab and Lapatinib treatments.

In various embodiments the WBP2 levels in the patient's sample are measured with at least one probe adapted to target a WBP2 protein.

In various embodiments the probe is an antibody. In various embodiment the antibody binds to or engages the WBP2 protein set forth in SEQ ID NO. 1 and/or compounds bind to or engage with sections of WBP2 protein set forth in SEQ ID NO. 1. Exemplary antibodies include polyclonal, monoclonal, humanized, bispecific, and heteroconjugate antibodies. Methods of making antibodies are known in the art. In various embodiments the antibody was generated to the epitope comprising the amino acid sequence set forth in SEQ ID NO. 2: NH2-NDMKNVPEAFKGTKKGT-COOH.

In various embodiments the probe is an aptamer. In various embodiments the aptamer comprises oligonucleotides binds to or engages the WBP2 protein set forth in SEQ ID NO. 1 and/or bind to or engage with sections of WBP2 protein set forth in SEQ ID NO. 1 and/or bind to or engage with sections of WBP2 peptide set forth in SEQ ID NO. 2.

In various embodiments the probe is a peptide. In various embodiments the peptide comprises amino acids that bind to or engage the WBP2 protein set forth in SEQ ID NO. 1 and/or bind to or engage with sections of WBP2 protein set forth in SEQ ID NO. 1 and/or bind to or engage with sections of WBP2 peptide set forth in SEQ ID NO. 2. In various embodiments Examples of the peptides include: amino acid sequence set forth in SEQ ID NO. 3: PPGYPPPYPPPY or amino acid sequence set forth in SEQ ID NO. 4:

YVQPPPPPYPGPMEPPVSGPDVPSTPAAEAKAAEAAASAY.

As used herein the term ‘cancer’ refers to any cancer involving abnormal cell proliferation. In various embodiments the cancer is a cancer where HER2 is overexpressed. In various embodiments the cancer is breast cancer, or ovarian cancer, or stomach cancer, or gastric cancer or uterine cancer or cholangiocarcinoma. In various embodiments the cancer is breast cancer.

In various embodiments the method is an in vitro method.

In various other embodiments the method is an in vivo method.

Another aspect of the invention provides a kit for identifying in a sample the amount of human epidermal growth factor receptor 2 (HER2) and the amount of WW domain-binding protein 2 (WBP2), the kit comprising: (a) at least one first probe adapted to detect and measure a human epidermal growth factor receptor 2 (HER2) level in the sample to determine whether the sample is HER2 positive or HER2 negative; and (b) at least one second probe adapted to detect and measure WW domain-binding protein 2 (WBP2) levels in the sample.

In various embodiments the first probe is adapted to target a HER2 gene. Any HER2 gene probe known in the art would be suitable.

In various embodiments the second probe is adapted to target a WBP2 gene. Wherein the WBP2 gene comprises a nucleic acid sequence set forth in SEQ ID NO. 5: aatgacatgaagaacgtgccagaagccttcaaagggaccaagaaaggc actgtctaccttaccccttaccgggtcatcifictgtccaagggcaaggatgccatgcagtcc or any segment thereof or complementary sequence thereof.

In various embodiments the first probe is adapted target a HER2 protein. In various embodiments the HER2 protein comprises amino acid sequence set forth in SEQ ID NO. 6:

MELAALCRWGLLLALLPPGAASTQVCTGTDMKLRLPASPETHLDMLRHLY
QGCQVVQGNLELTYLPTNASLSFLQDIQEVQGYVLIAHNQVRQVPLQRLR
IVRGTQLFEDNYALAVLDNGDPLNNTTPVTGASPGGLRELQLRSLTEILK
GGVLIQRNPQLCYQDTILWKDIFHKNNQLALTLIDTNRSRACHPCSPMCK
GSRCWGESSEDCQSLTRTVCAGGCARCKGPLPTDCCHEQCAAGCTGPKHS
DCLACLHFNHSGICELHCPALVTYNTDTFESMPNPEGRYTFGASCVTACP
YNYLSTDVGSCTLVCPLHNQEVTAEDGTQRCEKCSKPCARVCYGLGMEHL
REVRAVTSANIQEFAGCKKIFGSLAFLPESFDGDPASNTAPLQPEQLQVF
ETLEEITGYLYISAWPDSLPDLSVFQNLQVIRGRILHNGAYSLTLQGLGI
SWLGLRSLRELGSGLALIHHNTHLCFVHTVPWDQLFRNPHQALLHTANRP
EDECVGEGLACHQLCARGHCWGPGPTQCVNCSQFLRGQECVEECRVLQGL
PREYVNARHCLPCHPECQPQNGSVTCFGPEADQCVACAHYKDPPFCVARC
PSGVKPDLSYMPIWKFPDEEGACQPCPINCTHSCVDLDDKGCPAEQRASP
LTSIISAVVGILLVVVLGVVFGILIKRRQQKIRKYTMRRLLQETELVEPL
TPSGAMPNQAQMRILKETELRKVKVLGSGAFGTVYKGIWIPDGENVKIPV
AIKVLRENTSPKANKEILDEAYVMAGVGSPYVSRLLGICLTSTVQLVTQL
MPYGCLLDHVRENRGRLGSQDLLNWCMQIAKGMSYLEDVRLVHRDLAARN
VLVKSPNHVKITDFGLARLLDIDETEYHADGGKVPIKWMALESILRRRFT
HQSDWVSYGVTVWELMTFGAKPYDGIPAREIPDLLEKGERLPQPPICTID
VYMIMVKCWMIDSECRPRFRELVSEFSRMARDPQRFVVIQNEDLGPASPL
DSTFYRSLLEDDDMGDLVDAEEYLVPQQGFFCPDPAPGAGGMVHHRHRSS
STRSGGGDLTLGLEPSEEEAPRSPLAPSEGAGSDVFDGDLGMGAAKGLQS
LPTHDPSPLQRYSEDPTVPLPSETDGYVAPLTCSPQPEYVNQPDVRPQPP
SPREGPLPAARPAGATLERPKTLSPGKNGVVKDVFAFGGAVENPEYLTPQ
GGAAPQPHPPPAFSPAFDNLYYWDQDPPERGAPPSTFKGTPTAENPEYLG
LDVPV.

Any HER2 protein probe known in the art or able to bind to the HER2 protein would be suitable. In various embodiments the HER2 protein probe comprises an antibody. Table 2 lists the HER2 test kits currently approved by the USFDA.

TABLE 2
FDA-approved HER2 testing kits indicated as aids in the assessment
of patients for whom HER2-targeted treatment is being considered

| Assay type | Trade name | Manufacturer |
|---|---|---|
| Semi-quantitative IHC | HercepTest ™ | DAKO |
| IHC | PATHWAY ® | Ventana Medical |
| | | Systems Inc |
| IHC | InSite ® | Biogenex |
| | | Laboratories Inc |
| Semi-quantitative IHC | Bond Oracle ™ | Leica Biosystems |
| FISH | PathVysion ® | Abbott |
| | | Molecular Inc |
| FISH | PharmDx ™ Kit | DAKO |
| CISH | SPoT-Light ® | Life |
| | | Technologies Inc |
| CISH | INFORM HER2 | Ventana Medical |
| | dual ISH DNA | Systems Inc |
| | probe cocktail | |
| CISH | PharmDx ™ | DAKO |

CISH, chromogenic in situ hybridization; FDA, US Food and Drug Administration; FISH, fluorescence in situ hybridization; HER2, human epidermal growth factor receptor 2; IHC, immunohistochemistry; ISH, in situ hybridization.

In various embodiments the second probe is adapted to target a WBP2 protein. Wherein the WBP2 protein comprises an amino acid sequence set forth in SEQ ID NO. 1.

In various embodiments the first and second probe is an antibody. In various embodiments the antibody binds to or engages the WBP2 protein set forth in SEQ ID NO. 1 and/or bind to or engage with sections of WBP2 protein set forth in SEQ ID NO. 1 and/or bind to or engage with sections of WBP2 peptide set forth in SEQ ID NO. 2.

In various embodiments the first and second probe is an aptamer. In various embodiments the aptamer binds to or engages the WBP2 protein set forth in SEQ ID NO. 1 and/or bind to or engage with sections of WBP2 protein set forth in SEQ ID NO. 1 and/or bind to or engage with sections of WBP2 peptide set forth in SEQ ID NO. 2.

In various embodiments the first and second probe is a peptide. In various embodiments the peptide binds to or engages the WBP2 protein set forth in SEQ ID NO. 1 and/or bind to or engage with sections of WBP2 protein set forth in SEQ ID NO. 1 and/or bind to or engage with sections of WBP2 peptide set forth in SEQ ID NO. 2. In various embodiments the peptide comprises a sequence set out in any one of the peptide set forth in SEQ ID NO. 4 or SEQ ID NO. 5 or a fragment, homologue, variant or derivative thereof; or a polynucleotide comprising a nucleotide sequence that encodes any suitable polypeptide probe that binds to or engages the WBP2 protein set forth in SEQ ID NO. 1 and/or bind to or engage with sections of WBP2 protein set forth in SEQ ID NO. 1 and/or bind to or engage with sections of WBP2 peptide set forth in SEQ ID NO. 2. or a complement thereof

The terms mentioned in the kit are defined in a similar manner as the like terms mentioned above.

In various embodiments the kit further comprises written instructions for examining a sample to determine a prognosis of overall survival, cancer recurrence or response to cancer treatment for the patient.

In various embodiments the kit further comprises written instructions for calculating the predetermined level of HER2 and WBP2. In various embodiments the kit further comprises a device for calculating a prognosis of overall survival, cancer recurrence or response to cancer treatment for the patient based on the methods disclosed herein. In various embodiments the device includes a processor, a memory, a computer, a data base, a back end server, a communication network, a smart phone, a tablet, a handheld device an application on such a device or any similar device whereby the information such as details, data the level of HER2 and WBP2 and parameters measured with the kit can be included or entering and calculated to determine a prognosis of overall survival, cancer recurrence or response to cancer treatment for the patient based on the methods disclosed herein.

In various embodiments the kit further comprises components such as needle biopsy tools, vials, other equipment suitable for obtaining samples, and/or reagents for suitable detection.

Another aspect of the invention provides an in vitro method for determining the prognosis of overall survival, cancer recurrence or response to treatment, the method comprising: (a) measuring the level of human epidermal growth factor receptor 2 (HER2) in a sample; (b) measuring the level of WW domain-binding protein 2 (WBP2) in the sample, and (c) determining whether the level of HER2 and WBP2 are above or below a predetermined level wherein a result in step (c) provides a prognosis of overall cancer survival, cancer recurrence or response to cancer treatment.

Another aspect of the invention provides a method for the prognosis of survival or response to a treatment that targets EGFR or HER2 in a patient suffering from cancer comprising the steps of measuring WBP2 levels in a patient sample.

The terms mentioned in the in vitro method are defined in a similar manner as the like terms mentioned above. Similarly, all the steps mentioned and described above may be used with the in vitro method.

Throughout this document, unless otherwise indicated to the contrary, the terms “comprising”, “consisting of”, “having” and the like, are to be construed as non-exhaustive, or in other words, as meaning “including, but not limited to”.

Furthermore, throughout the specification, unless the context requires otherwise, the word “include” or variations such as “includes” or “including” will be understood to imply the inclusion of a stated integer or group of integers but not the exclusion of any other integer or group of integers.

As used in the specification, the singular form “a”, “an” and “the” include plural references unless the context clearly dictates otherwise.

Unless defined otherwise, all technical and scientific terms used herein have the same meaning as is commonly understood by a skilled person to which the subject matter herein belongs.

It should be further appreciated by the person skilled in the art that variations and combinations of features described above, not being alternatives or substitutes, may be combined to form yet further embodiments falling within the intended scope of the invention.

### Examples

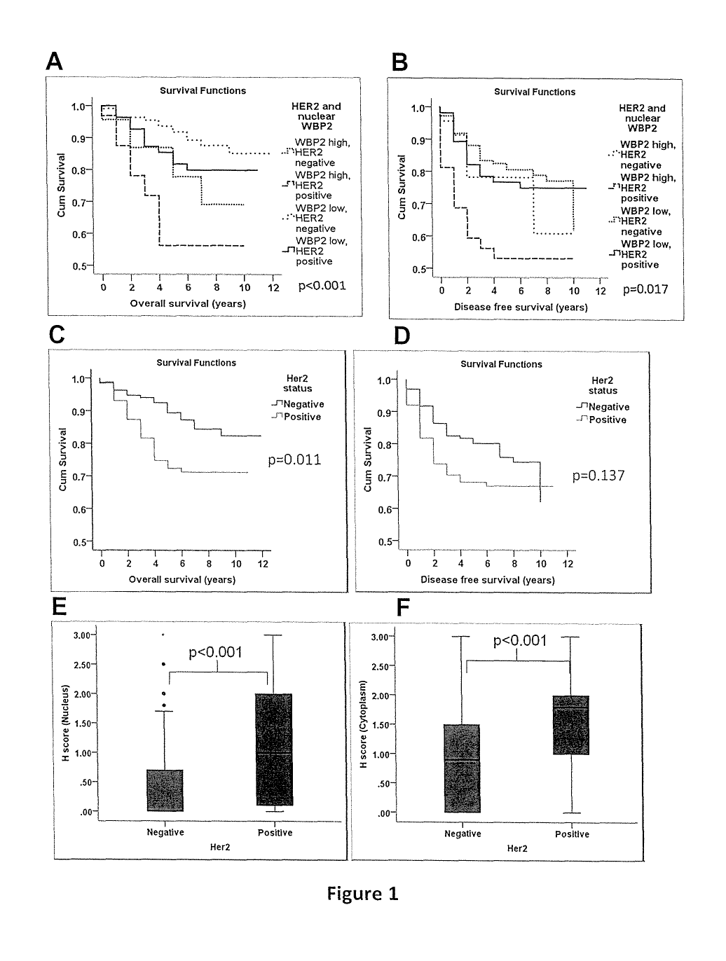

In an analysis involving more than 200 clinical specimens (n=221), WBP2 in combination with HER2 were discovered to be more powerful in predicting poorer overall survival and disease-free-survival than either WBP2 or HER2 alone (see Table 3 below and FIG. 1). With reference to only measuring stratification of HER2 positive and HER2 negative (FIGS. 1C and 1D) and measuring stratification of HER2 negative patients having low WBP2 levels, HER2 negative patients having high WBP2 levels, HER2 positive patients having low WBP2 levels, and HER2 positive patients having high WBP2 levels the differences seen are much greater. For example with reference to HER2 negative patients having low WBP2 levels the HER2 positive patients with high WBP2 levels have approximately 4.5 times lower chance of overall survival compared to approximately 1.7 times in HER2 positive patients with low WBP2 levels; and HER2 positive patients with high WBP2 levels have approximately 2.6 times higher chance of recurrence compared to approximately 1.1 times in HER2 positive patients with low WBP2 levels (FIGS. 1A and 1B). There is a correlation between HER2 and WBP2 overexpression and between HER2 and WBP2 normal expression levels (FIGS. 1E and 1F).

Value proposition: 1) WBP2 can be used to stratify HER2 positive breast cancers into lowly and highly aggressive cases for treatment and surveillance; 2) WBP2 confers aggression to HER2+ cases and predicts response to HER2 antagonist treatment which include but is not limited to Herceptin (Trastuzumab), Pertuzumab and Lapatinib treatments. An IHC score of more than 1 indicates high WBP2 levels while an IHC score of 1 or less indicates low WBP2 levels.

TABLE 3

| | Hazard | | |
|---|---|---|---|
| Markers | ratio | 95% C.I | P value |
| Overall survival | | | |
| Nuclear WBP2 low, HER2− | | | |
| as reference | | | |
| Nuclear WBP2 Low, HER2+ | 1.697 | 0.760-3.792 | 0.197 |
| Nuclear WBP2 high, HER2− | 2.446 | 0.929-6.440 | 0.070 |
| Nuclear WBP2 high, HER2+ | 4.494 | 2.107-9.584 | <0.001 |
| Disease-free survival | | | |
| Nuclear WBP2 low, HER2− | | | |
| as reference | | | |
| Nuclear WBP2 Low, HER2+ | 1.093 | 0.564-2.116 | 0.793 |
| Nuclear WBP2 high, HER2− | 1.437 | 0.619-3.335 | 0.399 |
| Nuclear WBP2 high, HER2+ | 2.583 | 1.353-4.930 | 0.004 |

### Antibodies

Through NeoMPS, Inc, we generated in-house polyclonal antibodies against WBP2 based on a 17 amino acid set for the in SEQ ID NO. 2 (N′-NDMKNVPEAFKGTKKGT-C′) peptide sequence, which were affinity purified and stringently validated via comparative immunoblotting with pre-immune serum, in the presence of WBP2-specific and control peptides, reciprocal immunoprecipitation of exogenously expressed tagged WBP2 protein and immunoblotting with anti-tag and anti-WBP2 antibodies (data—not shown). anti-PY20-HRP, were obtained from BD-Biosciences, San Diego, Calif., USA. Anti-HER2 antibodies are known in the art and may be obtained from any of the registered diagnostic kits available. For the current studies the HER2 diagnostic kit was obtained from the HER2 diagnosis kit from Roche Molecular Systems Inc. USA.

### Specimens

221 clinical specimens that included original resections and follow-up biopsies at time points after the original resection were collected from several hospitals in Singapore over a large timeframe with consent.

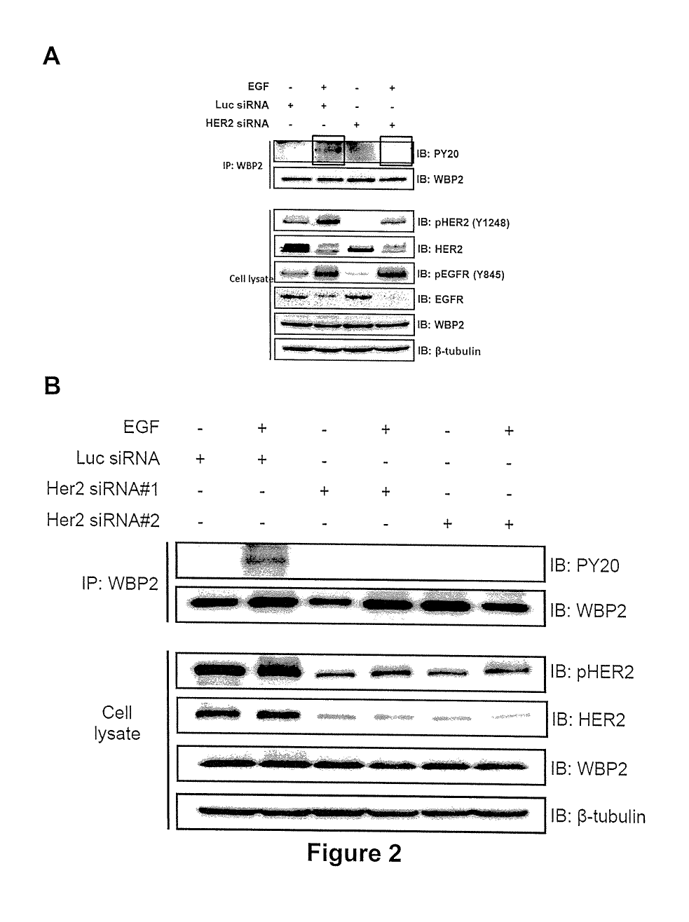

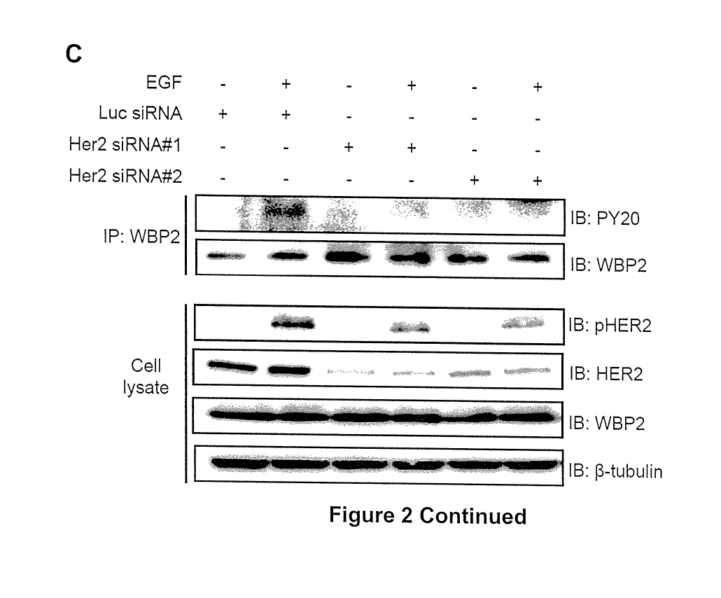

In an immunoblot analysis (FIG. 2A), WBP2 is shown to mediate EGF/HER2 signalling and WBP2 is shown to be a potential predictor of response to drugs that target EGFR/HER2. HER2 was knocked down in human breast cancer SK-BR-3 cells by transfection of HER2 siRNA. Luciferase siRNA was used as negative control. Cells were treated with 50 ng/ml EGF for 10 min after 24 hr serum starvation. Cell lysates were immunoprecipitated (IP) with anti-WBP2 antibody and phosphorylation of endogenous WBP2 were analysed by Western blot (IB) using anti-phosphotyrosine (PY20). Phosphorylation of HER2 and EGFR were analysed by Western blot (IB) with indicated antibodies. β-tubulin was used as a protein loading control.

Phosphorylation of WBP2 appears to depend on HER2 expression. Where HER2 was knocked down in human breast cancer cells, SK-BR-3 (FIG. 2B) and ZR-751(FIG. 2C) by transfection of HER2 siRNA using Luciferase siRNA as negative control the phosphorylation of WBP2 increased. Cells were treated with 50 ng/ml EGF for 10 min after 24 hr serum starvation. Cell lysates were immunoprecipitated (IP) with anti-WBP2 antibody and phosphorylation of endogenous WBP2 were analyzed by Western blot (IB) using anti-phosphotyrosine (PY20) and anti-WBP2 antibodies. Phosphorylation of HER2 was analysed by Western blot (IB) with indicated antibodies. β-tubulin was used as protein loading control.

In Vitro Models

Three separate breast cancer cell lines were examined, BT-474 human breast carcinoma cells characterized by the overexpression of HER2, SK-BR-3 breast cancer cells and breast cancer cell line ZR-75-30. Cells were treated with different concentration of Trastuzumab (0, 1, 10, 100 μg/ml) for 3 days (SK-BR-3) or 5 days (BT-474 and ZR-75-30).

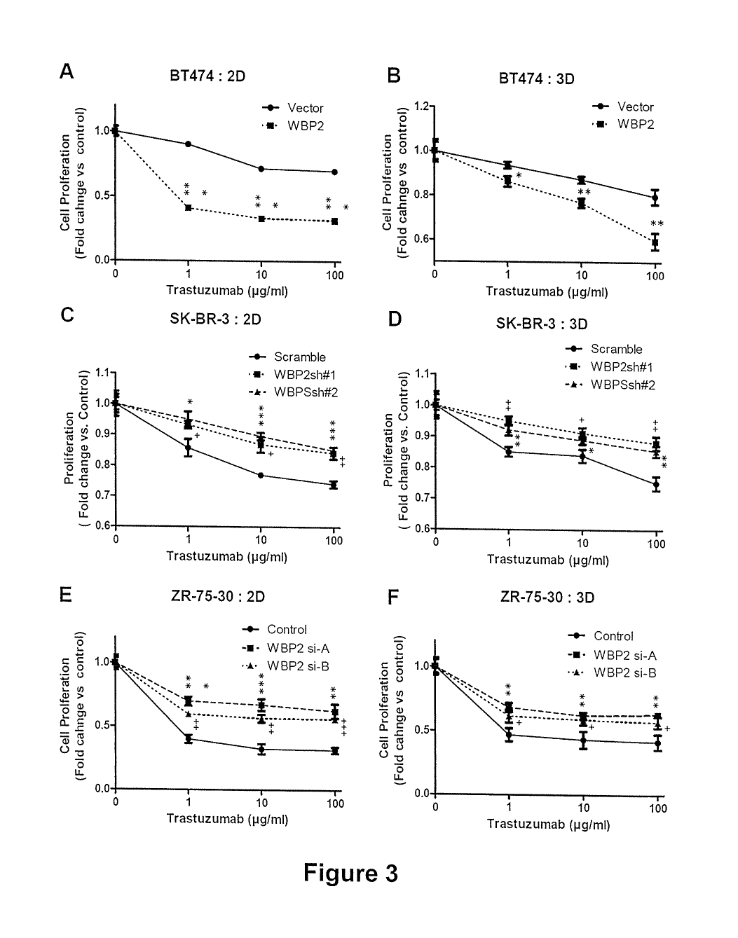

Cell that were overexpressing both WBP2 and HER2 were more sensitive to an HER2 antagonist such as Trastuzumab. Trastuzumab dose-response with WBP2 expression level effect cell proliferation. WBP2 was overexpressed in BT-474 human breast carcinoma cells characterized by the overexpression of HER2 using WBP2 expressing lentivirus resulting in a greater reduction of cell proliferation when treated with Trastuzumab compared to BT-474 human breast carcinoma cells characterized by the overexpression of HER2 with no enhanced WBP2 expression (FIGS. 3A and 3B). Similarly where WBP2 was knocked-down using two different shRNA targeting WPB2 in SK-BR-3 breast cancer cells the Trastuzumab treatment was less successful at reducing cell proliferation (FIGS. 3C and 3D). Again when two different siRNA targeting WBP2 where used in the breast cancer cell line ZR-75-30 the Trastuzumab treatment was less successful at reducing cell proliferation (FIGS. 3E and 3F). Cells were plated on 96-well plates for 2D culture (A and C) or 96-well ultra-low attachment plates for 3D culture (B and D) at 10,000 cells per well. After 3 days (SK-BR-3) or 5 days (BT-474) incubation with Trastuzumab, the viability of cells were measured by using Cell Titre 96 aqueous non-radioactive cell proliferation assay. Viability of cells was calculated as fold change compared to Trastuzumab-untreated control cells. The data represent mean±SD. Statistical significance was determined by Student's t-test (* or +P<0.05; ** or ++P<0.01; *** or +++P<0.001 vs. vector or control).

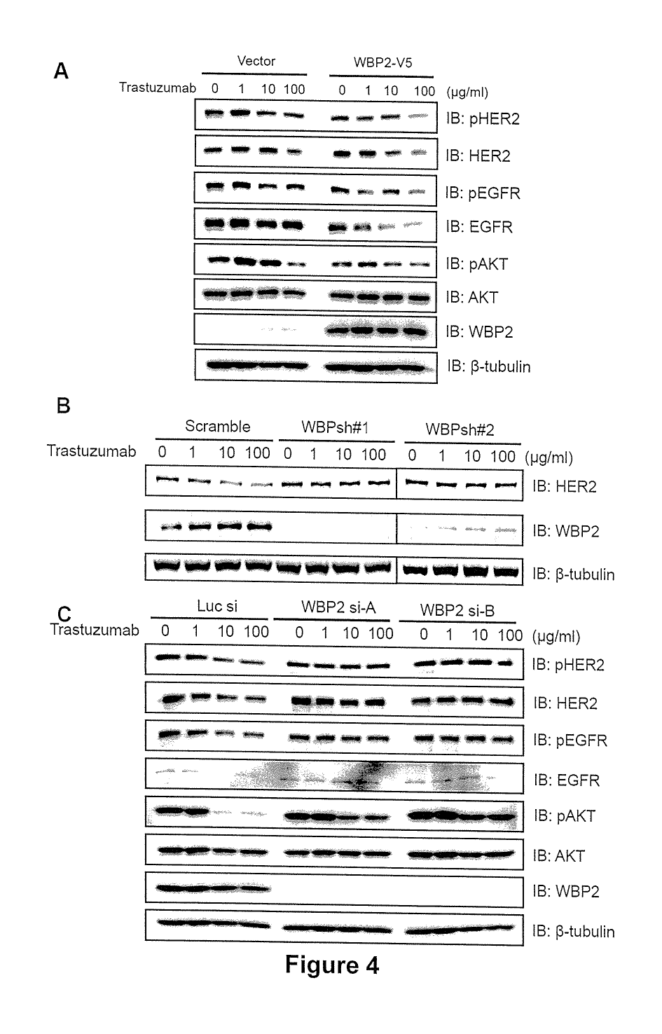

The protein expression profile during the Trastuzumab dose-response experiments listed above and in FIG. 3 demonstrated a similar pattern. When WBP2 was over expressed the expression levels of HER2 was more sensitive to HER2 antagonists in comparison to when the WBP2 expression was knocked down. When WBP2 was overexpressed in BT-474 human breast carcinoma cells characterized by the overexpression of HER2 using WBP2 expressing lentivirus (FIG. 4A) phosphorylated HER2, HER2, phosphorylated EGFR, EGFR and phosphorylated AKT were all reduced at higher dosages of Trastuzumab treatment. Contrastingly when WBP2 was knocked down using two different shRNA targeting WPB2 in SK-BR-3 breast cancer cells there was little change in the HER2 expression (FIG. 4B). Where WBP2 was knocked down using two different siRNA targeting WBP2 in ZR-75-30 breast cancer cells only reductions in phosphorylated AKT were observed at higher dosages of Trastuzumab treatment (FIG. 4C).

In Vivo Models

All animal housing and handling procedures were in accordance with institutional guidelines at National University of Singapore. For the Xenograft model, 5-week-old female Athymic Nude mice (n=6-7, In Vivos, Singapore) were implanted with 0.72 mg 60 day release 17β-estradiol pellets (Innovative Research, Sarasota, Fla., USA) and, after 2 days, BT-474 control (vector) or WBP2 overexpressing cells (1×107 in 200 μl of DPBS and Matrigel 1:1 mixture) were injected subcutaneously into a mouse mammary fat pad. BT-474 human breast carcinoma are characterized by the overexpression of human epidermal growth factors receptors 2 (HER-2) and estrogen receptors (ER). BT-474 cells grow in response to estradiol. Estradiol supplement is required to establish xenograft model of athymic nude mice.

When the tumours reached 150-200 mm3, the mice were divided into groups, keeping average tumour size similar between groups, and treated with Trastuzumab (10 mg/kg, Roche) or PBS (control) by intraperitoneally (IP) twice weekly for three weeks. The tumour size was measured twice weekly with callipers and tumour volumes calculated as follow: volume=(width2×length)/2. The data represent mean±SEM. Statistical significance was determined by Mann-Whitney test.

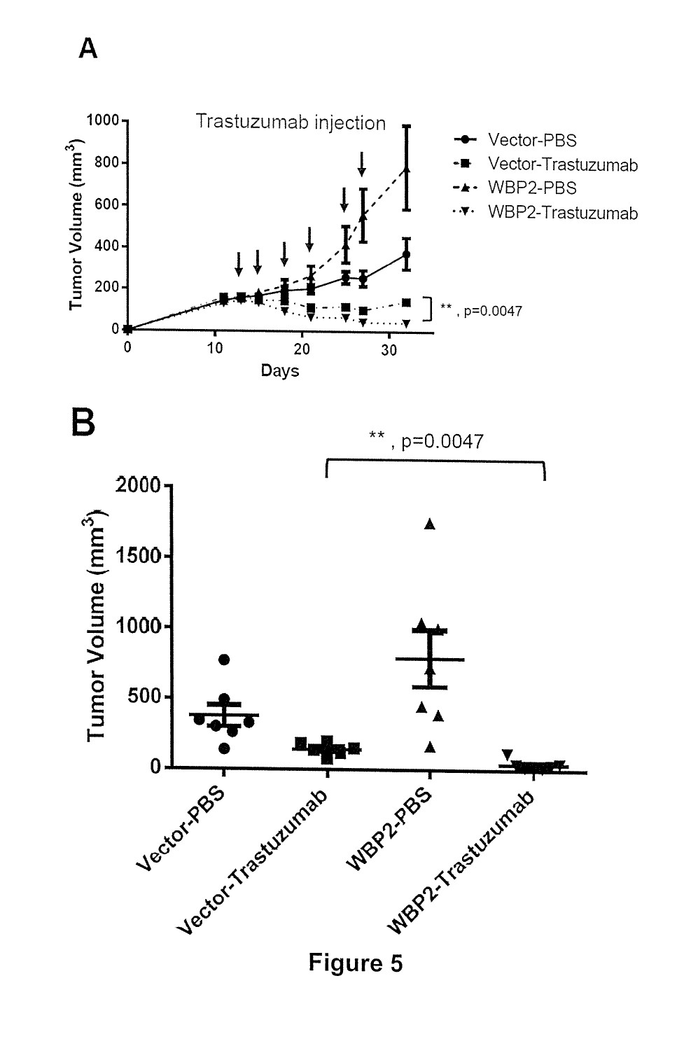

The response in the tumour volume is plotted over the 35 day treatment (FIG. 5A). Where it can be seen that tumours induced with the BT-474 human breast carcinoma increased or reduced in size when they were treated with PBS or Trastuzumab respectively. A similar trend was observed in tumours induced with WBP2 overexpressing cells. Each time point is depicted in FIG. 5B from where the mean tumour volume size and SEM statistical significance for each treatment was calculated. The end point data analysis is summarized in Table 4.

TABLE 4
End point data analysis of the tumour volume

| Tumour | Vector- | Vector- | WBP2- | WBP2- |
|---|---|---|---|---|
| volume | PBS | Trastuzumab | PBS | Trastuzumab |
| Mean | 376.7 | 144.2 | 791.2 | 41.36 |
| SEM | 76.57 | 18.46 | 201.3 | 13.5 |
| Trastuzumab | 100% | 38.28% | 100% | 5.23% |
| effect (% of | | | | |
| control) | | | | |

Similar results were obtained with patient derived xenograft models (PDX) whereby the PDX models included: Group 1 WBP2 low, HER2 negative PDX models; Group 2 WBP2 Low, HER2 positive PDX models; group 3 WBP2 high, HER2 negative; and group 4 WBP2 high, HER2 positive. Each group was treated with Trastuzumab (10 mg/kg, Roche) or PBS (control) intraperitoneally (IP) and the tumor size was measured twice weekly with calipers and tumor volumes calculated as follow: volume=(width²×length)/2. Group 2 had a larger average tumour volume than the group 4 (data not shown).

Those skilled in the art will appreciate that the invention described herein is susceptible to variations and modifications other than those specifically described. The invention includes all such variation and modifications. The invention also includes all of the steps, features, formulations and compounds referred to or indicated in the specification, individually or collectively and any and all combinations or any two or more of the steps or features.

Each document, reference, patent application or patent cited in this text is expressly incorporated herein in their entirety by reference, which means that it should be read and considered by the reader as part of this text. That the document, reference, patent application or patent cited in this text is not repeated in this text is merely for reasons of conciseness.

Any manufacturer's instructions, descriptions, product specifications, and product sheets for any products mentioned herein or in any document incorporated by reference herein, are hereby incorporated herein by reference, and may be employed in the practice of the invention.

The present invention is not to be limited in scope by any of the specific embodiments described herein. These embodiments are intended for the purpose of exemplification only. Functionally equivalent products, formulations and methods are clearly within the scope of the invention as described herein.

The invention described herein may include one or more range of values (e.g. size, concentration, etc.). A range of values will be understood to include all values within the range, including the values defining the range, and values adjacent to the range which lead to the same or substantially the same outcome as the values immediately adjacent to that value which defines the boundary to the range.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of the invention.

Other definitions for selected terms used herein may be found within the detailed description of the invention and apply throughout. Unless otherwise defined, all other scientific and technical terms used herein have the same meaning as commonly understood to one of ordinary skill in the art to which the invention belongs.

1. A method for the prognosis of overall survival, cancer recurrence or response to treatment for a patient suffering from cancer, the method comprising:
(a) examining a sample from the patient to determine whether the patient is human epidermal growth factor receptor 2 (HER2) positive or negative based on a predetermined level of HER2; and
(b) measuring WW domain-binding protein 2 (WBP2) levels in the patient's sample,
wherein a result in step (a) and a result in step (b) provides a prognosis of overall survival, cancer recurrence or response to treatment for the patient.

2. The method of claim 1, wherein the results in steps (a) and (b) are compared to a set of predetermined expression level results from a comparison population.

3. The method of claim 2, wherein the comparison population is stratified into a plurality of subgroups determining the aggressiveness of a cancer.

4. The method of claim 3, wherein each subgroup is referenced from a reference group of HER2 negative patients comprising WBP2 expression below a predetermined level.

5. The method of claim 4, wherein a sample with WBP2 expression levels above the predetermined level and a HER2 positive patient provides the prognosis that the patient has an approximate 4 to 5 times lower chance of overall survival compared to the reference group.

6. The method of claim 4, wherein a sample with WBP2 expression levels below the predetermined level and a HER2 positive patient provides the prognosis that the patient has an approximate 1 to 2 times lower chance of overall survival compared to the reference group.

7. The method of claim 4, wherein a sample with WBP2 expression levels above the predetermined level and a HER2 negative patient provides the prognosis that the patient has an approximate 2 to 3 times lower chance of overall survival compared to the reference group.

8. The method of claim 4, wherein a sample with WBP2 expression levels above the predetermined level and a HER2 positive patient provides the prognosis that the patient has an approximate 2 to 3 times higher chance of cancer recurrence compared to the reference group.

9. The method of claim 4, wherein a sample with WBP2 expression levels below the predetermined level and a HER2 positive patient provides the prognosis that the patient has an approximate 1 times higher chance of cancer recurrence compared to the reference group.

10. The method of claim 4, wherein a sample with WBP2 expression levels above the predetermined level and a HER2 negative patient provides the prognosis that the patient has an approximate 1 to 2 times higher chance of cancer recurrence compared to the reference group.

11. The method of claim 1, wherein a sample with WBP2 expression levels above the predetermined level and a HER2 positive result predicts that the patient is likely to respond to treatment.

12. The method of claim 1, wherein a sample with WBP2 expression levels below the predetermined level and a HER2 positive result predicts that the patient is less likely to respond to treatment.

13. The method of claim 11, wherein the treatment comprises a HER2 antagonist.

14. The method of claim 13, wherein the HER2 antagonist comprises Herceptin (Trastuzumab), Pertuzumab, Lapatinib, Lapatinib in combination with capecitabine, Trastuzumab emtansin, Ado-trastuzumab, Neratinib, Amrubicin or Dasatinib.

15. The method of claim 1, wherein the WBP2 levels in the patient's sample are measured with at least one probe adapted to target a WBP2 protein.

16. The method of claim 15, wherein the probe is selected from an antibody.

17-18. (canceled)

19. The method of claim 1, wherein the cancer is breast cancer.

20. The method of claim 1, wherein the method is an in vitro method.

21. (canceled)

22. A kit for identifying in a sample the amount of human epidermal growth factor receptor 2 (HER2) and the amount of WW domain-binding protein 2 (WBP2), the kit comprising:
(a) at least one first probe adapted to detect and measure a human epidermal growth factor receptor 2 (HER2) level in the sample to determine whether the sample is HER2 positive or HER2 negative; and
(b) at least one second probe adapted to detect and measure WW domain-binding protein 2 (WBP2) levels in the sample.

23. The kit of claim 22, wherein the first probe is adapted to target a HER2 gene or a HER2 protein.

24. The kit of claim 22, wherein the second probe is adapted to target a WBP2 gene or a WBP2 protein.

25-26. (canceled)

27. The kit of claim 25, wherein the first and second probe is selected from an antibody;
an aptamer; and
a peptide.

28-29. (canceled)

30. The kit claim 22, the kit further comprises written instructions for examining a sample to determine a prognosis of overall survival, cancer recurrence or response to treatment for the patient.

31. An in vitro method for determining the prognosis of overall survival, cancer recurrence or response to treatment, the method comprising:
(a) measuring the level of human epidermal growth factor receptor 2 (HER2) in a sample;
(b) measuring the level of WW domain-binding protein 2 (WBP2) in the sample; and
(c) determining whether the level of HER2 and WBP2 are above or below a predetermined level wherein a result in step (c) provides a prognosis of overall cancer survival, cancer recurrence or response to cancer treatment.

32. (canceled)


SEQUENCE LISTING (6 sequences)

SEQ ID NO: 1 - protein, 261 aa, Homo sapiens
MALNKNHSEGGGVIVNNTESILMSYDHVELTFNDMKNVPEAFKGTKKGTVYLTPYRVIFLSKGKDAMQSFMMPFYLMKDCEIKQPVFGANYIKGTVKAEAGGGWEGSASYKLTFTAGGAIEFGQRMLQVASQASRGEVPSGAYGYSYMPSGAYVYPPPVANGMYPCPPGYPYPPPPPEFYPGPPMMDGAMGYVQPPPPPYPGPMEPPVSGPDVPSTPAAEAKAAEAAASAYYNPGNPHNVYMPTSQPPPPPYYPPEDKKTQ

SEQ ID NO: 2 - protein, 17 aa, Homo sapiens
NDMKNVPEAFKGTKKGT

SEQ ID NO: 3 - protein, 12 aa, Homo sapiens
PPGYPPPYPPPY

SEQ ID NO: 4 - protein, 40 aa, Homo sapiens
YVQPPPPPYPGPMEPPVSGPDVPSTPAAEAKAAEAAASAY

SEQ ID NO: 5 - DNA, 111 bp, Homo sapiens
aatgacatgaagaacgtgccagaagccttcaaagggaccaagaaaggcactgtctaccttaccccttaccgggtcatctttctgtccaagggcaaggatgccatgcagtcc

SEQ ID NO: 6 - protein, 1255 aa, Homo sapiens [sequence omitted]